(19) United States
(12) Patent Application Publication
(10) Pub. No.: US 2002/0123804 A1
(43) Pub. Date: Sep. 5, 2002

(54) Scleral implants for treating presbyopia and methods for implanting the same

(75) Inventors: Arlene Gwon, Newport Beach, CA (US); Daniel G. Brady, San Juan Capistrano, CA (US)

Correspondence Address: Frank J. Uxa, Stout, Uxa, Buyan & Mullins, LLP, Suite 300, 4 Venture, Irvine, CA, 92618, US

(73) Assignee: Allergan Sales, Inc., Irvine, CA (US)

(21) Appl. No.: 09/797,243

(22) Filed: Mar. 1, 2001

(51) Int. Cl. A61F 2/14

(52) U.S. Cl. 623/4.1

(57) ABSTRACT

A scleral implant includes a body with a central portion and a pair of end portions. Each of the portions has a parametrical dimension. The parametrical dimension of the central portion is greater than the parametrical dimensions of each of the end portions. Accordingly, when the implant is positioned within an incision in the sclera of an eye, the inward force of the incision on the central portion of the body frictionally retains the implant within the incision. The scleral implant may include one or more enlarged portions each having a parametrical dimension greater than the parametrical dimension of the central portion. The implant may structure for engaging with sides of the incision such that extrusion of the implant from the incision is mitigated. The structure may include a plurality of dimples or a plurality of nodes formed in the surface of the body. Each of these structures forms a discontinuity or a purchase on the body to which post-operative cellular and fibrous growth may attached.

### BACKGROUND OF THE INVENTION

The present invention relates to the treatment of eye disorders such as presbyopia and, more particularly, relates to devices that are implanted in the sclera and associated methodology for implanting such devices.

Presbyopia is a vision condition in which the crystalline lens of an eye loses flexibility, making it difficult for the eye to focus on near objects. Presbyopia may seem to occur suddenly to a patient, but the actual loss of flexibility typically takes place over a number of years, and usually becomes noticeable in the early to mid-forties. Presbyopia is a natural part of the aging process of the eye and, therefore, is not a disease and cannot be prevented. Some signs of presbyopia include the tendency to hold reading materials at arm's length, blurred vision at normal reading distance, and eye fatigue along with headaches when doing close work. Comprehensive optometric examinations typically include testing for presbyopia.

To help compensate for presbyopia, optometrists prescribe reading glasses, bifocals, trifocals, or contact lenses. As presbyopia can complicate other common vision conditions like nearsightedness, farsightedness, and astigmatism, optometrists determine the specific lenses to allow clear and comfortable vision. Laser eye surgery cannot improve presbyopia.

Rather than simply compensating for presbyopia with eyeglasses, surgical procedures have been developed for the treatment of presbyopia. One such procedure is known as anterior ciliary sclerotomy (ACS). ACS is a surgical incisional technique used to correct up to +2.00 diopters (D) of presbyopia. The procedure is based on the use of eight or more radial incisions in the sclera over the ciliary body of the eye. The incisions produce more room for the lens to accommodate for near vision. This effectively increases focal power and focal depth. The incisions are placed beginning in the surgical limbus, extending 2 mm to 3 mm posteriorly and stopping just anterior to the pars plana of the eye. This procedure can be enhanced by adding more incisions or reversed by suturing previously placed incisions.

In another approach, the distance of the ciliary muscle from the lens equator is increased by implanting segmental polymethylmethacrylate (PMMA) scleral-expansion bands through scleral tunnels. Four scleral incisions at 2.75 mm posterior to the limbus are equidistantly placed 90 degrees from each other. The segmental scleral implants are then inserted through each scleral tunnel, or belt loop, to vault the ciliary muscle away from the lens equator.

One of the drawbacks of conventional ACS procedures is that the incisions heal and close, thereby untimely reversing the beneficial effects of the procedure. Accordingly, a longer-lasting surgical method for increasing increase accommodation of the eye for near vision is needed.

### SUMMARY OF THE INVENTION

The present invention provides new and enhanced implants and surgical methods for the treatment of presbyopia. The implants and related implantation methodology have a number of advantages over conventional approaches. For example, scleral incisions are inhibited from closing post-operatively which would otherwise reverse the beneficial effects of treatment procedures. In addition, the implants are substantially self-retaining within the incisions, thereby streamlining implantation procedures and decreasing trauma to the eye.

According to one aspect of the invention, a scleral implant includes a body with a central portion and a pair of end portions. Each of the portions has a parametrical dimension. The parametrical dimension of the central portion is greater than the parametrical dimensions of each of the end portions. Accordingly, when the implant is positioned within an incision in the sclera of an eye, the inward force of the incision on the central portion of the body frictionally retains the implant within the incision.

One of the advantages of the invention is that the relatively large central portion of the body enhances the retention of the implant within the incision. More specifically, when inserted within an incision, the implant forces the sides of the incision outwardly, thereby causing an opposing inward force upon the body, thereby retaining the implant within the incision. In addition, by separating the opposing sides of the incision, the implant inhibits the sclera surrounding the incision from healing and closing the incision, which would reverse the effects of the procedure.

According to another aspect of the invention, the scleral implant may include an enlarged portion that has a parametrical dimension greater than the parametrical dimension of the central portion. To an even greater degree than the central portion, the enlarged portion retains the implant within an incision and inhibits the sclera from closing the incision. In a preferred embodiment, the enlarged portion is disposed substantially equidistantly between the end portions and extends circumferentially around the body.

According to another aspect of the invention, the body includes a plurality of the enlarged portions. For example, the enlarged portions may be wing-like structures projecting outwardly from the body, thereby increasing the parametrical dimension of the central portion. Each of the enlarged portions engages with sides of the incision to increase retention therewithin.

According to still another aspect of the invention, an implant for treating presbyopia includes structure for enhancing post-operative cellular and fibrous growth-not to close the incision-but to further increase the retention of the implant within an incision. More specifically, a scleral implant includes a body configured for insertion into an incision made in the sclera of an eye. The body includes structure for engaging with sides of the incision such that extrusion of the implant from the incision is mitigated.

For example, the structure may include an enlarged portion for abutting the sides of the incision as discussed above. Alternatively, the structure may include a plurality of dimples formed in the surface of the body. Each of the dimples forms a discontinuity or a purchase on the body to which post-operative cellular and fibrous growth may attached. In still another embodiment, the structure may include a plurality of nodes protruding outwardly from the body. Like the dimples, each of the nodes forms a discontinuity for enhancing the attachment of post-operative cellular growth. Other embodiments of such structure include one or more through holes formed through the body of the implant.

Any number of configurations of the implant are possible. For example, the central portion of the body may be substantially cylindrical. In this embodiment, the parametrical dimensions of the central portion may have a diameter of less than about 0.6 mm. A ratio of a length of the implant to the parametrical diameter of the central portion may be less than about 8 to 1. Other geometries are possible, including polygonal cross sections such as triangular and square.

In addition to providing devices for the treatment of presbyopia, the present invention also provides methods for the treatment of presbyopia. According to the methodology of the invention, a plurality of implants are provided, each including a body with an enlarged portion. The body is configured for insertion into an incision made in the sclera of an eye. The enlarged portion is configured to engage with surfaces of the incision such that extrusion of the body from the incision is mitigated. The next step in the methodology is to make a plurality of incisions in the sclera of an eye. This incision may be made in accordance with any of the many known procedures in the art. An implant is then positioned within each of the incisions, for example, so that the enlarged portion thereof engages with surfaces of the incision.

One of the advantages of the treatment methods of the invention is that the incisions are prevented from closing which would otherwise reverse the beneficial effects of the procedure. More specifically, the implants serve as spacers or expanders with inserted into the incisions.

Another advantage of the methodology is that the implants are substantially self-retaining within the incisions. By urging the sides of the incisions apart, the resulting inward forces of the surrounding sclera frictionally retain the implants within respective incisions.

Any and all of the features described herein and combinations of such features are included within the scope of the present invention provided that the features of any such combination are not mutually inconsistent.

Additional aspects, features, and advantages of the present invention are set forth in the following description and claims, particularly when considered in conjunction with the accompanying drawings in which like parts bear like reference numbers.

### BRIEF DESCRIPTION OF THE DRAWINGS

FIG. 1 is a plan view of a scleral implant shown in accordance with an exemplary embodiment of the present invention, particularly illustrating a scleral implant configured for treating presbyopia;

FIG. 2 is a cross-sectional view of the scleral implant of FIG. 1 taken along line 2-2, particularly illustrating a central portion of the implant and a corresponding parametrical dimension;

FIG. 3 is a cross-sectional view of the scleral implant of FIG. 1 taken along line 3-3, particularly illustrating an end portion of the implant and a corresponding parametrical dimension that is less than that of the central portion;

FIG. 4 is a plan view of a scleral implant shown in accordance with another exemplary embodiment of the present invention;

FIG. 5 is a cross-sectional view of an eye, illustrating incisions made in the sclera for receiving implants of the invention for the treatment of presbyopia;

FIG. 6 is a front plan view of the cornea and sclera an eye, illustrating the incisions made in the sclera for receiving implants of the invention for the treatment of presbyopia;

FIG. 7 is a plan view of an implant of the present invention positioned within an incision made in the sclera of an eye;

FIG. 8 is a plan view of a scleral implant shown in accordance with yet another exemplary embodiment of the present invention, particularly illustrating enlarged mid-portion for engaging sides of an incision;

FIG. 9 is a cross-sectional view of the scleral implant of FIG. 8 taken along line 9-9;

FIG. 10 is a plan view of a scleral implant shown in accordance with still another exemplary embodiment of the present invention, particularly illustrating a pair of diametrically opposed enlarged mid-portions;

FIG. 11 is a cross-sectional view of the scleral implant of FIG. 10 taken along line 11-11;

FIG. 12 is a plan view of the implant of FIG. 10 positioned within an incision made in the sclera of an eye;

FIG. 13 is a cross-sectional view of the scleral implant of FIG. 10 positioned in an incision and taken along line 13-13 of FIG. 12;

FIG. 14 is a plan view of a scleral implant shown in accordance with a further exemplary embodiment of the present invention, particularly illustrating a thread-type enlarged portion;

FIG. 15 is a cross-sectional view of the scleral implant of FIG. 14 taken along line 15-15;

FIG. 16 is a plan view of a scleral implant shown in accordance with a still further exemplary embodiment of the present invention, particularly illustrating a plurality of enlarged mid-portions;

FIG. 17 is a plan view of a scleral implant shown in accordance with still another exemplary embodiment of the present invention, particularly illustrating a noncylindrical implant;

FIG. 18 is a cross-sectional view of the noncylindrical scleral implant taken along line 18-18 of FIG. 17;

FIG. 19 is an elevation view of the noncylindrical scleral implant taken along line 19-19 of FIG. 17;

FIG. 20 is a plan view of a scleral implant shown in accordance with still another exemplary embodiment of the present invention, particularly illustrating another noncylindrical implant;

FIG. 21 is a cross-sectional view of the noncylindrical scleral implant taken along line 21-21 of FIG. 20;

FIG. 22 is a plan view of a scleral implant shown in accordance with yet another exemplary embodiment of the present invention, particularly illustrating an implant with structure for enhancing the retention of post-operative cellular growth;

FIG. 23 is a plan view of a scleral implant shown in accordance with a further exemplary implant having structure for enhancing the retention of post-operative cellular growth;

FIG. 24 is a plan view of a scleral implant shown in accordance with a still further exemplary embodiment of the present invention, particularly illustrating an implant with a longitudinally extending through hole;

FIG. 25 is a cross-sectional view of the scleral implant taken along line 25-25 of FIG. 24; and

FIG. 26 is a plan view of a scleral implant shown in accordance with another exemplary embodiment of the present invention, particularly illustrating an implant with a plurality of through holes.

### DETAILED DESCRIPTION OF THE INVENTION

An exemplary implant 50 for an eye produced in accordance with the teachings of the present invention is illustrated in FIG. 1. For purposes of explanation and without limiting the scope of the present invention, exemplary implant 50 is illustrated as an implant for insertion into the sclera of an eye for the treatment of presbyopia. However, the principles of the present invention are applicable to other uses of the implant, which will become apparent from the following detailed description.

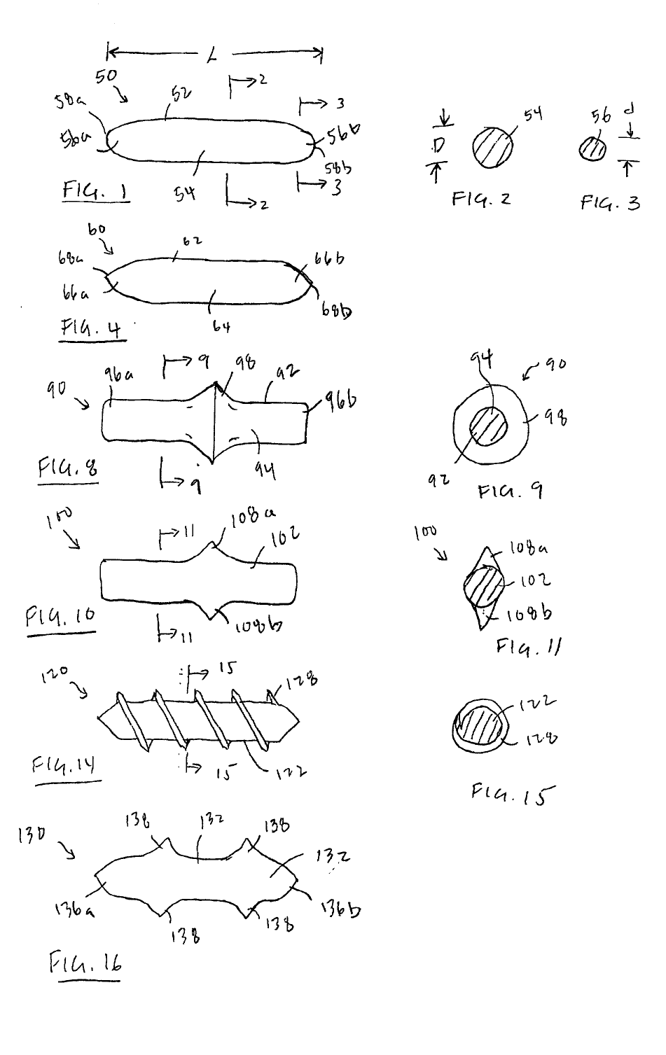

Exemplary implant 50 includes an elongate body 52 with a central portion 54 and a pair of end portions 56a and 56b each with an end 58a and 58b, respectively. As shown in FIG. 2, exemplary central portion 54 has a parametrical dimension, and as shown in FIG. 3, each end portion 56 has a parametrical dimension, with each dimension being defined as a distance around the periphery of a respective portion. The body 52 is made from a biocompatible material such as polymethylmethacrylate (PMMA), a polymeric material, or an acrylic material. It is preferable for the material to be fairly rigid, nonabsorbent, and nonbiodegradable.

According to the present invention, the parametrical dimension of the central portion 54 is larger than the parametrical dimension of each of the end portions 56. Therefore, the likelihood of extrusion or dislodgment of the implant from an incision in the sclera is minimized because of the non-uniform cross section. In addition, scleral incisions are inhibited from closure and subsequent healing by the implant 50. According to a preferred embodiment, the body 52 of exemplary implant 52 may be substantially cylindrical where the parametrical dimensions are circumferences based on a diameter D (FIG. 2) of the central portion 54 and a diameter d (FIG. 3) of the end portions 56.

An example of a commercial embodiment of the implant 50 configured for typical ACS procedures may have a longitudinal length L of the order of about 2 to 3 millimeters (mm) and a diameter D of less than about 0.5 mm. An exemplary ratio of length L to diameter D may be less than about 8 to 1, depending upon the procedure.

An alternative embodiment of an implant 60 of the invention is shown in FIG. 4. Exemplary implant 60 includes an elongate body 62 with a central portion 64 and a pair of end portions 66a and 66b, with the parametrical dimension of the central portion 64 being larger than the parametrical dimension of each of the end portions 66. In contrast to curved ends 58 of implant 50 shown in FIG. 1, ends 68 of implant 60 have pointed or bullet-shaped ends 68. In addition to a relatively large central portion 64, pointed ends 68 may enhance the implantation and retention of implant 60 within a scleral incision.

In this regard, reference is made to FIGS. 5 and 6 which illustrate an eye 70 including a cornea 72 and a sclera 74, which is the front part or anterior white of the eye. The eye 70 also includes a crystalline lens 76 supported by zonules 78 on a ciliary body 80. Conjunctiva 82 lies over the cornea 72 and a forepart of the sclera 74. For a detailed discussion on the anatomy of the eye, see, for example, GRAY'S ANATOMY, 24^{th} Edition (Lea & Febiger, 1942) , pages 1018 to 1037, the entire disclosure of which is incorporated herein by reference.

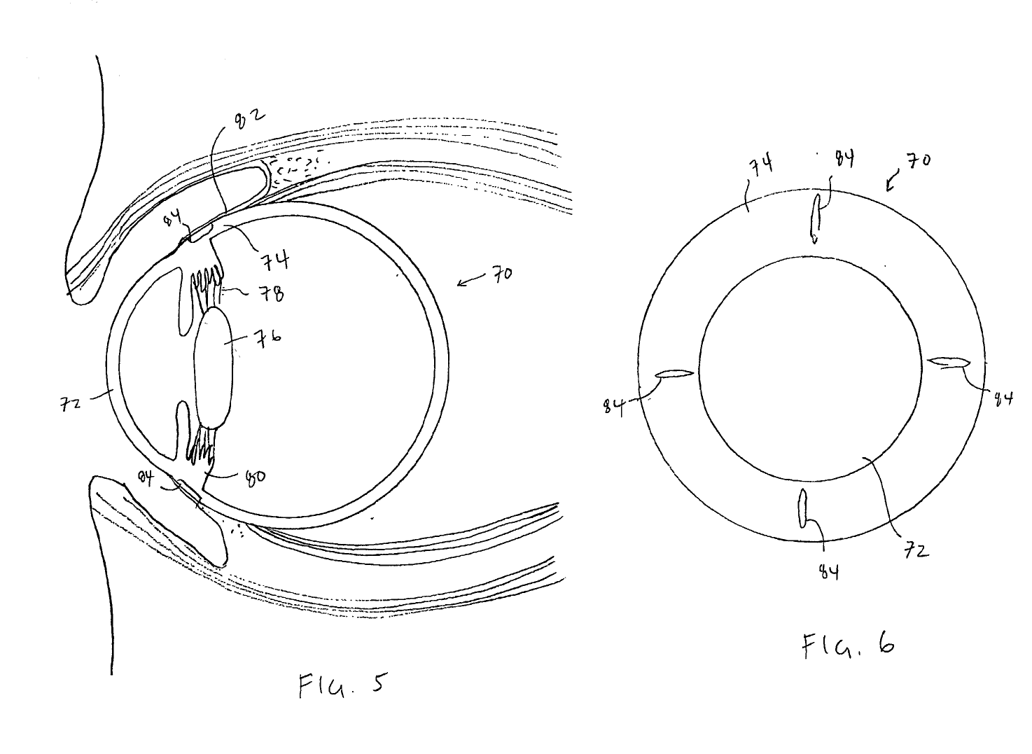

During anterior ciliary sclerotomy (ACS), a number of incisions 84 are made in the sclera 74, e.g., four to eight incisions typically. With reference to FIG. 7, according to the present invention, an implant 50 (or 60) is inserted into each incision 84 with the enlarged central portion 54 being subject to inward forces of the sclera 74, as indicated by arrows A, thereby retaining the implant 50 within the incision 84. The incisions 84 may be made according to conventional ACS procedures. With the implant 50 being securely retained within the incision 84, the sclera 74 surrounding the incisions 84 is inhibited from closing and thereby reversing the effect of the implantation procedure.

Referencing FIGS. 8 and 9, retention within an incision may be further facilitated with additional structure. For example, an exemplary implant 90 of the invention includes a body 92 with a central portion 94 and a pair of end portions 96a and 96b. In addition, implant 90 includes an enlarged portion 98, preferably formed or disposed at a location along the central portion 94. According to the invention, the enlarged portion 98 has a parametrical dimension that is larger than the parametrical dimension of the central portion 94 (as well as that of each of the end portions 96).

As shown in FIG. 9, the enlarged portion 98 may extend around the entire cross-sectional parameter of the body 92. In an alternative embodiment as shown in FIGS. 10 and 11, an implant 100 includes a body 102 with a plurality of enlarged portions (e.g., two) 108a and 108b. According to a preferred embodiment, the enlarged portions 108 are diametrically disposed on the body 102 as particularly shown in FIG. 11.

In implantation procedures as shown in FIGS. 12 and 13, the enlarged portions 108 engage with sides 110 of an incision 84, thereby inhibiting dislodgment of the implant 110. In addition, because of the enlarged parametrical dimension, the sclera 74 causes an inward force on the implant 100, as shown by arrows A. In a preferred embodiment, the enlarged portions 108 may be positioned along the body 102 substantially equidistantly between the ends of the implant 100.

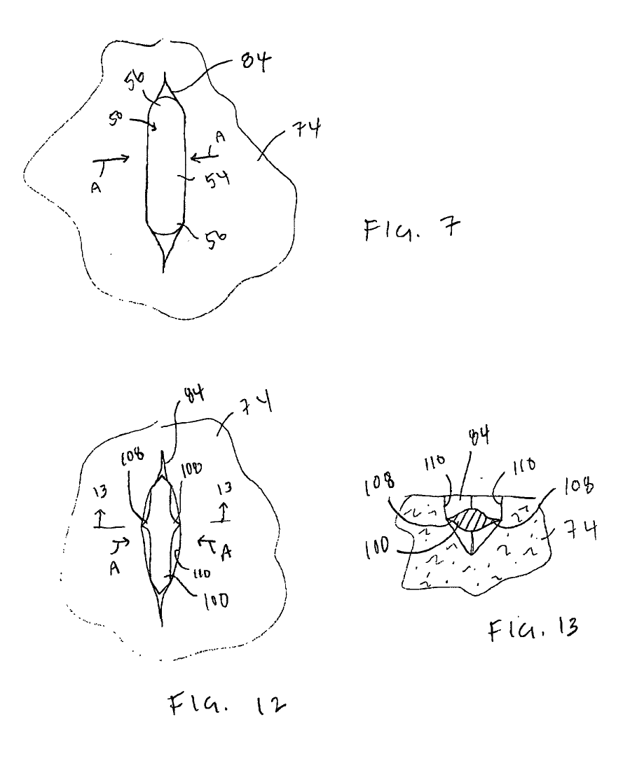

Referencing FIGS. 14 and 15, another embodiment of an implant 120 of the present invention includes a body 122 and an enlarged portion 128. According to this embodiment, the enlarged portion 128 is configured analogously to the threads of a screw, helically disposed about the body 102.

Another embodiment of an implant 130 of the invention is shown in FIG. 16 and includes a body 132 with end portions 136a and 136b and a plurality of enlarged portions 138 disposed along the body 102. The enlarged portions 138 are positioned at two locations at or near the end portions 136.

In addition to the substantially cylindrical configuration of the body described above, the implants of the present invention may be configured in alternative shapes. For example, as shown in FIGS. 17 and 18, an exemplary implant 140 includes a three-sided body 142 that is substantially triangular in cross section. End portions 146 of the body 142 may each terminate in a point as shown in FIG. 19.

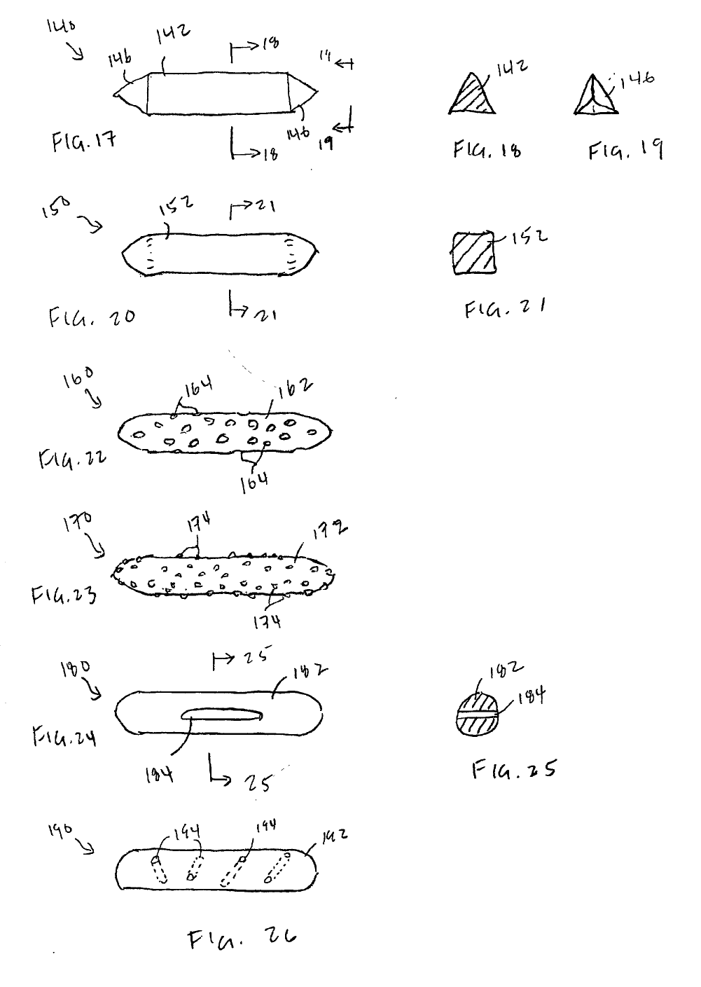

Alternatively, an exemplary implant 150 according to another embodiment of the invention is shown in FIGS. 20 and 21 and includes a four-sided body 152 that is substantially square in cross section.

As mentioned above, the implants of the invention include features that are designed to enhance the retention of the implant within an incision. In addition to the enlarged portions satisfying this purpose, the implants may include structure that further facilitates engagement with the sides of an incision. For example, as shown in FIG. 22, an exemplary implant 160 includes a body 162 with a plurality of dimples 164 formed in a surface thereof. Each of the dimples 164 defines a discontinuity in the surface of the body 162 that acts to create friction against the sides 110 of the incision 84. In addition, the dimples 162 provide a purchase for post-operative cellular and fibrous growth which may further enhance the retention of the implant 160 in an incision 84.

In another approach to providing a discontinuous surface for cellular and fibrous growth, an exemplary implant 170 of the invention shown in FIG. 23 includes a body 172 with a plurality of nodes 174 protruding outwardly therefrom. As with the dimples described above, each of the nodes 174 provides a discontinuity in the surface of the body 172 that acts to create friction against the sides of 110 of the incision 84 may engage. In addition, each of the nodes 174 may protrude slightly into the sclera 74 to enhance the frictional engagement with the sides 110 of an incision 84.

Yet another embodiment of an implant 180 of the invention is shown in FIGS. 24 and 25. Exemplary implant 180 includes a body 182 with a through hole 184. Analogous to the dimples and the nodes described above, the through hole 184 provides structure to which post-operative cellular and fibrous growth may grow, thereby securely retaining the implant 180 within an incision 84. According to a preferred embodiment, the through hole 184 extends longitudinally along the body 182 of the implant 180 as particularly shown in FIG. 24.

According to still another embodiment, an implant 190 may include a body 192 with a plurality of through holes 194 as shown in FIG. 26. The through holes 194 are preferably distributed along the longitudinal extent of the body 192 at varying angles.

While the present invention has been described with respect to various specific examples and embodiments, it is to be understood that the invention is not limited thereto and that it can be variously practiced within the scope of the following claims.

What is claimed is:

1. A scleral implant comprising:
a biocompatible body including a central portion and a pair of end portions sized to fit within a radial scleral incision formed to increase accommodation of the lens of an eye;
each of the portions having a parametrical dimension;
the parametrical dimension of the central portion being greater than the parametrical dimensions of each of the end portions.

2. The scleral implant of claim 1 wherein the body includes an enlarged portion having a parametrical dimension that is greater than the parametrical dimension of the central portion.

3. The scleral implant of claim 2 wherein the enlarged portion is disposed substantially equidistantly between the end portions.

4. The scleral implant of claim 2 wherein the body includes a plurality of the enlarged portions.

5. The scleral implant of claim 1 further comprising at least one dimple disposed on the body.

6. The scleral implant of claim 1 further comprising at least one node disposed on the body.

7. The scleral implant of claim 1 further comprising at least one hole formed through the body.

8. The scleral implant of claim 1 wherein the central portion is substantially cylindrical.

9. The scleral implant of claim 8 wherein the parametrical dimensions of the central portion is a diameter of less than about 0.6 mm.

10. An implant for treating presbyopia, the implant comprising:
a body including an enlarged portion;
the body being configured for insertion into an incision made in the sclera of an eye; and
the enlarged portion being configured to engage with surfaces of the incision such that extrusion of the body from the incision is mitigated.

11. The scleral implant of claim 10 wherein the body includes a plurality of enlarged portions.

12. The scleral implant of claim 10 wherein the enlarged portion is positioned on the body at a central location.

13. The scleral implant of claim 10 wherein the body is elongated.

14. The scleral implant of claim 10 wherein the body has a length and the enlarged portion has a parametrical diameter;
a ratio of the length to the parametrical diameter being less than about 8 to 1.

15. A method for treating presbyopia, the method comprising:
providing a plurality of implants each including a body;
the body being configured for insertion into an incision made in the sclera of an eye; and
the body being configured to engage with side surfaces of the incision such that extrusion of the body from the incision is mitigated;
making a plurality of incisions in the sclera of an eye; and
inserting one of the implants in each of the incisions.

16. The method of claim 15 wherein the body has at least one enlarged portion, and further comprising:
positioning each of the implants inserted into the incisions such that the enlarged portion thereof engages with surfaces of the incision.

17. An implant for treating presbyopia, the implant comprising:
a body configured for insertion into an incision made in the sclera of an eye;
the body including structure for engaging with sides of the incision such that extrusion of the implant from the incision is mitigated.

18. An implant of claim 17 wherein the structure includes an enlarged portion for abutting the sides of the incision.

19. An implant of claim 17 wherein the structure includes at least one dimple for forming a discontinuity on the body.

20. An implant of claim 17 wherein the structure includes at least one node protruding outwardly from the body.
